SEQUENCE LISTING (8 sequences)

SEQ ID NO: 1 - DNA, 1320 bp, Clostridium thermocellum
  source 1..1320: mol_type = genomic DNA
atgatggaatacttcaaaaatgtaccacaaataaaatacgaaggaccaaagtcaaacaacccatatgcatttaaattttacaatcctgatgaaataatagatggaaaacctttaaaagaacacttgcgtttttcagtagcgtactggcatacatttacagccaatgggacagatccatttggagcacctacaatgcaaagaccatggaatcatctaagtgatcctatggatattgctaaggcgagggtagaggcagcttttgaattttttgaaaagcttgacgtaccgtttttctgttttcatgacagagatatagctccagaaggggagaatttaagagagacgaacaaaaatttagatacaatagttgcaatgattaaagattatttaaagacgagcaaaacaaaagtattatggggtacagcaaatcttttttcaaatccgagatttgtacatggagcagcaacttcctgtaatgcagatgtatttgcatatgcagcagcccaagtaaaaaaagcacttgaaataacaaaagaacttggaggacagaactatgtattttggggcggaagagaaggatatgaaacactactcaatacagatatggaattggaacttgataatctcgcaagatttttgcacatggcagtagaatatgcaaaagagataggatttgaagggcagcttttaattgagccaaaaccaaaggaaccgacaaaacaccaatatgattttgacgcagcgaacgtatatgcatttttgaagaaatatgaccttgataaatacttcaaattaaacatagaagcaaaccatgccacacttgcaggacatgactttcaacatgaattaagatatgcaagaattaacaatatgttaggctctatagatgcaaatatgggagatatgcttttaggatgggatacagaccaatttccgacagacatacgaatgacaacccttgcaatgtatgaagtcattaagatgggtggttttgacaaaggtgggcttaattttgatgcgaaagtaagacgtgcttcatttgaaccagaagacctatttttagggcatattgcaggaatggatgcttttgtaaaaggatttaaagtagcgtataagcttgttaaagatggtgtatttgatagatttatagaagaaagatacaaaagttatcgagaaggcataggagcagagatagtaagtggaaaagcgaactttaaaactcttgaagaatatgcattaaacaatccaaagattgagaacaaatcaggcaagcaagagttgttagagtcaatattaaatcagtatttatttagtgaatga

SEQ ID NO: 2 - DNA, 1503 bp, Clostridium thermocellum
  source 1..1503: mol_type = genomic DNA
atgtattttcttgggatagatttgggtacatcagctgtaaagataattttaatagaggaaaagggaaatgtaataggaagcacatcaaaagaatatccagtatattaccctcagccaggctggtcagaacaaaatcctgaagactggtggaatgccacaaaagatggcatacgtgagttaataattaaaactggtgtaaaaaatgatgatataaaaggcataggtttaagtggccaaatgcatgggcttgtacttttagatgagaacaacaatgtactaatgcctgctatactttggaatgaccaaaggacgcaagaggagtgtggttatattacccaaacattaggcaaagaaagattgacaaaatatacagggaacaaagcattaacaggatttacagcgccaaagatattatgggtaaaaaaacatcgcactgatatatataaaaagattcatcatatacttttacctaaagattacatcagattcaaacttacaggggaatatgccacagatgtgtcagatgcatcaggtacattgttgtttgatgtagaaaacaggaaatggtcaaaagagatgttagatatattggacataccctataattggatgccaaaatgttatgaatctacggaggtaactggatatgtcacaaaagaggcagcggatttgacaggattaaaagaagggacaatagttgtaggcggaggaggagatcaagcaagtggagcagtagggactggtacagtaaagagtggcatagtatcagttgcacttggcacttcaggagttgtatttgcaagtcaagacaaatatgtagtagatgaagaaaacagattacactctttctgtcatgctaatggcaaatggcatgtaatgggagtaatgctctcagcagccgcttgtttaaaatggtggatagataacataatcaactttaatggttcttctataacatatgaaaagcttttagaagaagcagaaaaagtaacaccaggaagcggtgggttaatatttctcccttatctcatgggggaaaggactcctcatagtgacccttacgcgagaggatgctttatagggcttaatatgactcacaatagaggacatatgacaagagcaatacttgaaggggtagcatttggacttagagactcgcttgagataataaaagaacttgagataccagtaaatgaagttagagtaagcggcggcggtgcaaagagcaaattgtggaggcaaatacttgcagacatttttggtgtcagagtagacatggtaaatgcgactgaagggccagcttttggcgcagcaataatggcagcagtgggatatggaatatttaaagatgttgaggaggcatgtagtacactcatcaaagtgactgatagtgtttacccaataggagaaattgtaagtaaatataatgaaatatatcagatttatagaggtttgtataaggcccttaaagataggtttggggaaatagccagtataaattaa

SEQ ID NO: 3 - DNA, 2042 bp, Clostridium thermocellum
  source 1..2042: mol_type = genomic DNA
gtgataagtaaatctttttatgcgcatcacagcgcatttggcgctttctcaagttttgtaatcggtaaatgcggtaaaggcggtggcgtcgtactaaatgatgttcggccgcctgaaaacaacgtctacattggatacaaaagagatggtgttataagcttgctgccatttattaaagatgatacaaaaaatgctgaagaagagtttacaggagaagtctctacaagcaaaaaagaaaaaaacataaaaatctttggggaagatgagatagaaagagagttatgctgggcatcagacacttggacagcaggagacttcaaattttccatcatcactccatttggatacgtaaaagatccttcggtgatgaatggagacgaaaagaaacttgcactggcacctgtcatatttgtacagttgacaatggataatactgacagcgataaggatgctgagatgatatttggctttgaaggtccgaaaaggatattatctgagcttacagatggaaaatacttaggaggagtatacggcagaaaatacggttttgctatcaaaaaaagcgatgatgtaagagagctttcaaggcttgatattttgacatcatgggcaaatgacaactatcaaaatcatgggcttggcagagcgccgtctttgatatttaaagtgccgagaggggagaaaaggacatatactgtggcattggcaacgtatcaaagcggcgtcataacaacaggaatcgatgctgaattttactacacatctgtttttaagtcattggaagaagtattatcctttggacttgacaatcaagattattacttaaatttagcaaaggaaagagatgaagagcttaagaaaagcggtttaaatgaatacaggcagtttttattggcacatgcagcccacagttactatgccagcacggagcttttaaagagagacgtggtatgcctctttgggtggtaaacgaaggcgaatacattatgataaatacatttgatttgacggttgatcatgtcttctgggaaatgaggttccatccttggacgattacaaatacattggatctgtactatgaaaagtacagctacagggatcaagcaggtcttgcctttacgcatgatatgggtgtcgcagatggtttttctaaagaaggctattcatcttacgagcttccaaacctgactggatgttttagctacatgacacatgaggagcttttgaattgggttttgacaggttctgtctatgcaataaaaataaatgataaagaatggttaaagaaaaacatgggtgtattcgaagattgtttcgattctcttgtggcaagagataaaaataatgatggaataatggacgttgacagttcaaggtgtgagacggggtcggaaataacgacttacgatagccttgacgaaagcttgggacaggcgagaaacaatctataccttggtgttaagacatgggcagcttacgtgatgttgcatggtttgtttaaagaaaatgatcttagtgaaaaggcagaaaaagctttagaaaaggcaagacaggctgctaatactatcgttgccaagtttgacgaagaaaatcagtatatacctgcagtatttgagaatggcaacacatcaaggataatacctgctgtagaggcattggtatatccatatgttgtaggatatactgactttgtaagtgaagatggtgtatttggtgggcttataaaagccttaaagaagcatgtaatgacgattatgaagcctggtatatgcatagatgaagtatctggaggttggaagctttcgtcaaccagcaagaacacatggaatagtaaaattttcttatgccaatatgtgataaaagatgtgcttaatatagactttggagacaaagagattgagtgggacaaagtacacgcaatgtggcaacaggtgtcttgcagtgaagattgcgctacagatcaggtaaacagcgatacaggtacgccaagaggaagccgcttgtatccgagacttgtgacaagtgtattgtggatgaaatag

SEQ ID NO: 4 - DNA, 20 bp, synthetic construct
  source 1..20: mol_type = other DNA
agatgacgtaagctttatcg

SEQ ID NO: 5 - DNA, 23 bp, synthetic construct
  source 1..23: mol_type = other DNA
gcacttggcacttcaggagttgt

SEQ ID NO: 6 - DNA, 20 bp, synthetic construct
  source 1..20: mol_type = other DNA
cttgagaaagcgccaaatgc

SEQ ID NO: 7 - DNA, 20 bp, synthetic construct
  source 1..20: mol_type = other DNA
tcgtcaaccagcaagaacac

SEQ ID NO: 8 - DNA, 20 bp, synthetic construct
  source 1..20: mol_type = other DNA
tttctcctcatccgctcctg

(19) United States
(12) Patent Application Publication
(10) Pub. No.: US 2025/0257369 A1
(43) Pub. Date: Aug. 14, 2025

(54) ENGINEERED CLOSTRIDIUM THERMOCELLUM FOR CO-UTILIZATION OF HEMICELLULOSE AND CELLULOSE

(71) Applicant: Alliance for Sustainable Energy, LLC, Golden, CO (US)

(72) Inventors: Katherine Jenyan CHOU, Lakewood, CO (US); Trevor James CROFT, Golden, CO (US); Pin-Ching MANESS, Golden, CO (US); Wei XIONG, Golden, CO (US); Luis Humberto REYES BARRIOS, Bogatá (CO); Eric Ryan SCHAEDIG, Denver, CO (US); Jianping YU, Golden, CO (US); Skyler D. HEBDON, Golden, CO (US)

(21) Appl. No.: 19/018,909

(22) Filed: Jan. 13, 2025

Related U.S. Application Data
(60) Provisional application No. 63/620,497, filed on Jan. 12, 2024.

(51) Int. Cl. C12P 3/00; C12N 9/24; C12N 15/74

(52) U.S. Cl. CPC C12P 3/00; C12N 9/2402; C12N 15/74; C12Y 302/01037; C12N 2800/101

(57) ABSTRACT

Disclosed herein are engineered C. thermocellum strains capable of degrading and assimilating hemicellulose polysaccharide while retaining their cellulolytic capabilities that enable the immense potential of consolidated bioprocessing of biomass for an improved bioeconomy.

### CROSS-REFERENCE TO RELATED APPLICATIONS

This application claims priority under 35 U.S.C. § 119 to U.S. Provisional Patent Application No. 63/620,497 filed on 12 Jan. 2024, the contents of which are hereby incorporated in their entirety.

### CONTRACTUAL ORIGIN

This invention was made with government support under Contract No. DE-AC36-08GO28308 awarded by the Department of Energy. The government has certain rights in the invention.

### SEQUENCE LISTING

The instant application contains a Sequence Listing which has been submitted electronically and is hereby incorporated by reference in its entirety. The XML copy as filed herewith was originally created on 5 May 2025 is named NREL 24-02.xml and is 13 kilobytes in size.

### BACKGROUND

There is a need for highly efficient biomass deconstruction at high loadings of biomass in a bioreactor, utilization and conversion of the biomass to a product that is produced at high titer and productivity, which remains challenging. There is also a need for a biocatalyst (i.e., bacteria) to reduce or eliminate potentially inhibitory biomass/feedstock degradation intermediates. Co-culture of bacterial species to achieve consolidated bioprocessing (CBP) in its own appears to be a short-term solution and may complicate the issues-to-be-resolved by introducing a second species into the system.

### SUMMARY

In an aspect, the provided methods and systems describe methods for making and compositions of matter for engineered Clostridium thermocellum capable of co-utilizing cellulose and hemicellulose. In an embodiment, the engineered Clostridium thermocellum is capable of simultaneous co-utilization of cellulose and xylan. In another embodiment, the engineered Clostridium thermocellum comprises β-xylosidase which reduces xylan degradation intermediates comprising xylose and xylobiose.

In an aspect, disclosed herein is a method for making a second-generation biofuel from the consolidated bioprocessing of biomass using an engineered Clostridium thermocellum capable of co-utilizing cellulose and hemicellulose. In an embodiment, the biofuel is hydrogen.

Without being bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 depicts a summary of adaptive laboratory evolution (ALE) performed followed by subsequent strain characterization. A lineage of genome integrated xylAB (KJC335) was evolved in rich medium supplied with xylose (5 g/L) as the main carbon source. One other lineage of the integrated strain was evolved initially on xylose but later split in its evolutionary course in xylan (5 g/L) supplemented with diminishing amounts of xylose (1.8 g/L followed by 0.7 g/L). “RM” abbreviates for rich medium.

FIGS. 2A and 2B depict a comparison of growth on xylose and cellobiose (5 g/L of either sugar) across evolved mutants, KJC19-1, KJC19-9, KJCXn4, and KJCXn6, to the parental strain KJC335, and the minimally engineered strain Dhpt as a reference. FIG. 2A depicts clonal isolates KJC19-1, KJC19-9, KJCXn4, and KJCXn6 selected with only xylose reached higher growth rates than the unevolved KJC335 strain on xylose and is near the growth rate displayed by Dhpt on cellobiose, which is the bacteria's naturally preferred substrate. KJC19-1 and KJC19-9 growth on xylose also outperformed KJCXn4 and KJCXn6 which were selected with xylan and xylose combined. FIG. 2B depicts KJC19-9, KJC19-1, KJCXn4, and KJCXn6 appeared to have better retained their ability to grow on cellobiose. Data are reported as average±stdev (n=3).

FIGS. 3A, 3B depict that adaptive laboratory evolution (ALE) has substantially improved the engineered strain's growth on xylose (5 g/L). FIG. 3A depicts the time required to reach the maximal optical density (OD at 600 nm) dropped considerably within the first 6 transfers, while the maximal culture density reached by the evolved mixtures gradually increased. FIG. 3B depicts growth rates on xylose (5 g/L) of selective sub-cultures descending from KJC335 was increased during the first six transfers. No notable improvements were seen past the 14^{th} transfer. Precautions were taken to ensure that the cultures were transferred at late-log phase and before the stationary phase to avoid mutations arisen due to stress induced by nutrient depletion.

FIGS. 4A, 4B, 4C depict engineered cells carrying plasmids with various T. saccharolyticum β-xylosidase genes to improve polysaccharide utilization were grown on xylan (5 g/L). FIG. 4A depicts KJC19-9 with plasmid containing xylD, pKJC155-38, grows to a higher optical density compared KJC19-9 with empty vector, pKJC84. The xylC and xylB containing strains grew similar to empty vector. FIG. 4B depicts polysaccharides from corn core were quantified using acid hydrolysis and HPLC. Improved xylan utilization by KJC19-9 with pKJC155-38 (xylD) was observed. FIG. 4C depicts the ability to utilize xylan resulted in higher production and yield of H₂ gas. Data is reported as average±stdev (n=3). The p values are calculated using two-tailed, paired Student's t test (*, p<0.05).

FIGS. 5A, 5B, 5C, 5D, 5E, 5F, 5G, 5H, 5I depict co-utilization of cellulose (2.5 g/L) as Avicel and xylan (2.5 g/L) as xylan from corn core was achieved by KJC19-9 with pKJC155-38 (xylD). FIG. 5A depicts the cellulose solids from the Avicel were quickly degraded or utilized by all strains. FIG. 5b depicts that xylan was minimally detected in the solids fraction because xylan from corn core is soluble. FIG. 5C depicts that few glucan residues were detected in the soluble fraction indicating cellulose derived oligomers from the solids are quickly consumed by C. thermocellum. FIG. 5D depicts the xylD expressing strain fully degrades xylan by 11 hours. (E) Minimal glucose is produced during deconstruction. FIG. 5F depicts the xylose concentration increased and peaked at 11 hours in the xylD expressing strain, which is followed by xylose consumption. FIG. 5G depicts the total glucan and glucose saccharides are similarly consumed by all three strains. FIG. 5H depicts only KJC19-9 with pKJC155-38 can consume xylan from corn core. FIG. 5I depicts the xylD expressing strain produces up to 50% more H₂ gas than Δhpt or KJC19-9 with empty vector. Data is reported as average±stdev (n=2 for Δhpt or KJC19-9 with empty vector pKJC84, and n=4 for KJC19-9 with pKJC155-38).

FIGS. 6A, 6B, 6C, 6D, 6E depict KJCBXint outperforms KJC19-9 due to its ability to further deconstruct and consume the hemicellulose portion of deacetylated mechanically refined (DMR) corn stover loaded at ˜5 g/L as cellulose. FIG. 6A depicts KJC19-9 and KJCBXint solubilized similar amounts of solid biomass. The pink bars represent DMR composition before fermentation. The green and blue bars represent composition at the end of fermentation. FIG. 6B depicts that less than 0.3 g/L of soluble sugars remained as polysaccharides greater than 2 sugars in length. FIG. 6C depicts that KJC19-9 has innate ability to degrade xylan polysaccharides into xylobiose, but only KJCBXint can finish the depolymerization of xylobiose into xylose monomers. FIG. 6D depicts that based on total saccharides that sum up sugars in solid and soluble fractions, KJCBXint has the ability to consume xylose derived from hemicellulose. FIG. 6E depicts KJCBXint produced 20% more H₂ than KJC19-9. Data is reported as average±stdev (n=3) from pH-controlled fermentation in bioreactors. The p values are calculated using two-tailed, paired Student's t test (*, p<0.05).

FIG. 7 depicts hydrogen production by engineered organisms disclosed herein for various biomass feedstocks including Avicel, corn stover, dairy fiber, hard almond shells, soft almond shells, mint slug, coffee chaff, Sargassum and no biomass.

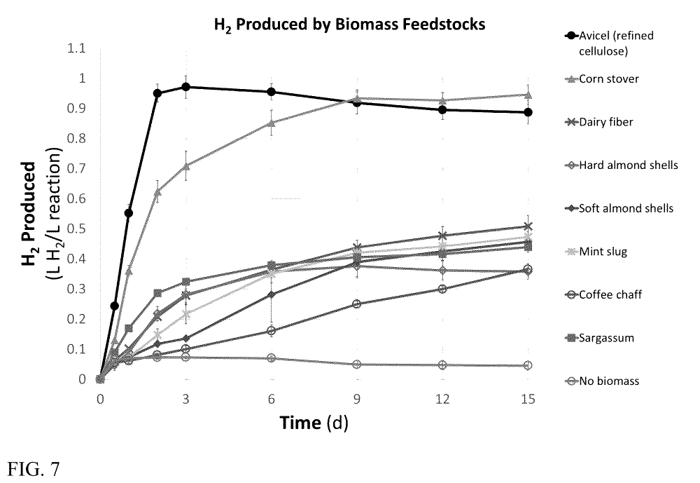

FIG. 8 depicts a proof-of-principle demonstrating that adaptive laboratory evolution (ALE) improved the growth of an engineered strain that recombinantly expressed xylAB genes on a plasmid. Growth is measured in CTFUD rich medium supplemented with 5 g/L of xylose as the main carbon source. Strain LH33202 is a colony isolated from a mixture of evolved mutants after nearly 100 generations (doublings) post selection in 5 g/L of xylose. The lag phase was reduced from over 25 hours for the parental culture to ˜11 hours in the evolved mutant. Data is reported as average±stdev (n=2).

FIGS. 9A, 9B, depict (FIG. 9A) clonal isolates, KJCXn4 and KJCXn6, undergone ALE in xylan supplemented with xylose, were only able to grow to low culture density when xylan (5 g/L) was supplemented with xylose (1.8 g/L). FIG. 9B depicts that supplementation with xylose nearly doubled the culture density but is substantially lower than the growth level reached with cellobiose (5 g/L) by Δhpt or by KJC355 with xylose (5 g/L). Data is reported as average±stdev (n=3).

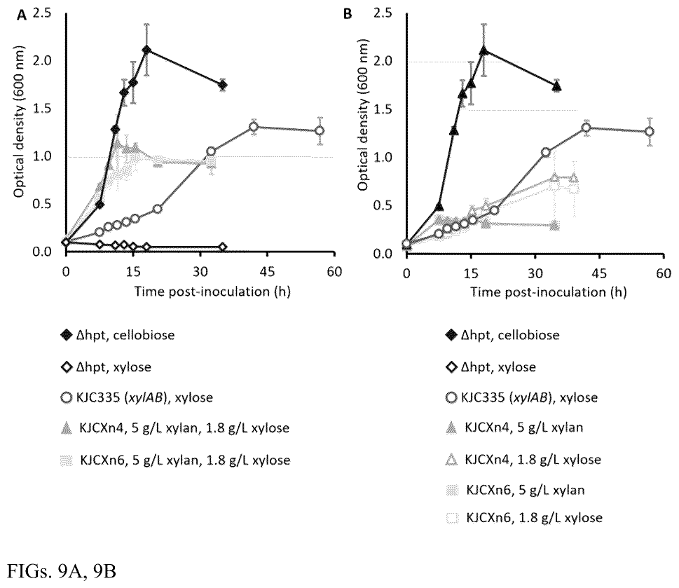

FIG. 10 depicts the xylD gene from T. saccharolyticum was integrated into the KJC19-9 xylose-evolved strain, after xylAB that resides at the old hpt locus. PCR on the gDNA was done to confirm presence of xylD in the correct orientation. The 7 kb fragment that was generated using primers 143 and 287 based on “BXint 4 gDNA” was sent for sequencing. The positive strain was name KJCBXint. FIG. 10 lists the sequences of primers used herein including primer 143 (SEQ ID NO: 4), primer KC8-4 (SEQ ID NO: 5), primer 210 (SEQ ID NO: 6), primer 217 (SEQ ID NO: 7), and primer 287 (SEQ ID NO: 8).

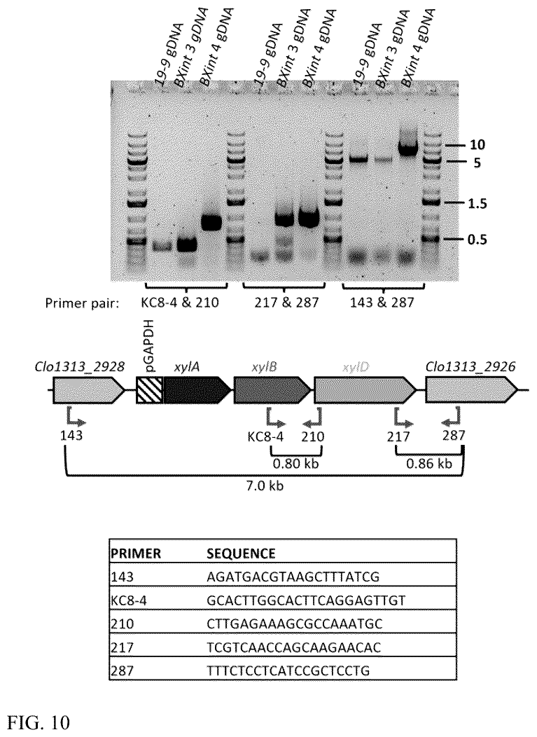

FIG. 11 depicts the sequence of the 7 kb fragment amplified by gDNA from KJCBXint using primers 143 and 287. Non-bold black represents flanking regions corresponding of Clo1313_2927. Bolded black represents the GAPDH promoter that was integrated and drives expression of the downstream xylA gene (SEQ ID NO: 1), in blue, and xylB gene (SEQ ID NO: 2), in green. The xylD gene (SEQ ID NO: 3) is highlighted in orange. Between the three genes is a non-bolded black text that represents ribosome binding sites.

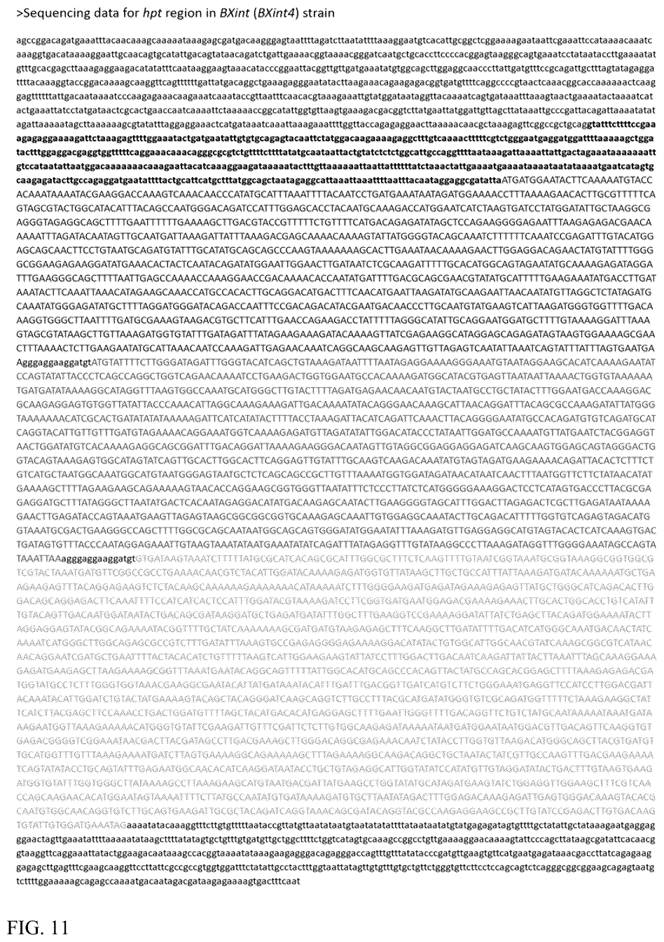

is:
SEQ ID NO: 1
ATGATGGAATACTTCAAAAATGTACCACAAATAAAATACGAAGGACCAAA
GTCAAACAACCCATATGCATTTAAATTTTACAATCCTGATGAAATAATAG
ATGGAAAACCTTTAAAAGAACACTTGCGTTTTTCAGTAGCGTACTGGCAT
ACATTTACAGCCAATGGGACAGATCCATTTGGAGCACCTACAATGCAAAG
ACCATGGAATCATCTAAGTGATCCTATGGATATTGCTAAGGCGAGGGTAG
AGGCAGCTTTTGAATTTTTTGAAAAGCTTGACGTACCGTTTTTCTGTTTT
CATGACAGAGATATAGCTCCAGAAGGGGAGAATTTAAGAGAGACGAACAA
AAATTTAGATACAATAGTTGCAATGATTAAAGATTATTTAAAGACGAGCA
AAACAAAAGTATTATGGGGTACAGCAAATCTTTTTTCAAATCCGAGATTT
GTACATGGAGCAGCAACTTCCTGTAATGCAGATGTATTTGCATATGCAGC
AGCCCAAGTAAAAAAAGCACTTGAAATAACAAAAGAACTTGGAGGACAGA
ACTATGTATTTTGGGGCGGAAGAGAAGGATATGAAACACTACTCAATACA
GATATGGAATTGGAACTTGATAATCTCGCAAGATTTTTGCACATGGCAGT
AGAATATGCAAAAGAGATAGGATTTGAAGGGCAGCTTTTAATTGAGCCAA
AACCAAAGGAACCGACAAAACACCAATATGATTTTGACGCAGCGAACGTA
TATGCATTTTTGAAGAAATATGACCTTGATAAATACTTCAAATTAAACAT
AGAAGCAAACCATGCCACACTTGCAGGACATGACTTTCAACATGAATTAA
GATATGCAAGAATTAACAATATGTTAGGCTCTATAGATGCAAATATGGGA
GATATGCTTTTAGGATGGGATACAGACCAATTTCCGACAGACATACGAAT
GACAACCCTTGCAATGTATGAAGTCATTAAGATGGGTGGTTTTGACAAAG
GTGGGCTTAATTTTGATGCGAAAGTAAGACGTGCTTCATTTGAACCAGAA
GACCTATTTTTAGGGCATATTGCAGGAATGGATGCTTTTGTAAAAGGATT
TAAAGTAGCGTATAAGCTTGTTAAAGATGGTGTATTTGATAGATTTATAG
AAGAAAGATACAAAAGTTATCGAGAAGGCATAGGAGCAGAGATAGTAAGT
GGAAAAGCGAACTTTAAAACTCTTGAAGAATATGCATTAAACAATCCAAA
GATTGAGAACAAATCAGGCAAGCAAGAGTTGTTAGAGTCAATATTAAATC
AGTATTTATTTAGTGAATGA.
is:
SEQ ID NO: 2
ATGTATTTTCTTGGGATAGATTTGGGTACATCAGCTGTAAAGATAATTTT
AATAGAGGAAAAGGGAAATGTAATAGGAAGCACATCAAAAGAATATCCAG
TATATTACCCTCAGCCAGGCTGGTCAGAACAAAATCCTGAAGACTGGTGG
AATGCCACAAAAGATGGCATACGTGAGTTAATAATTAAAACTGGTGTAAA
AAATGATGATATAAAAGGCATAGGTTTAAGTGGCCAAATGCATGGGCTTG
TACTTTTAGATGAGAACAACAATGTACTAATGCCTGCTATACTTTGGAAT
GACCAAAGGACGCAAGAGGAGTGTGGTTATATTACCCAAACATTAGGCAA
AGAAAGATTGACAAAATATACAGGGAACAAAGCATTAACAGGATTTACAG
CGCCAAAGATATTATGGGTAAAAAAACATCGCACTGATATATATAAAAAG
ATTCATCATATACTTTTACCTAAAGATTACATCAGATTCAAACTTACAGG
GGAATATGCCACAGATGTGTCAGATGCATCAGGTACATTGTTGTTTGATG
TAGAAAACAGGAAATGGTCAAAAGAGATGTTAGATATATTGGACATACCC
TATAATTGGATGCCAAAATGTTATGAATCTACGGAGGTAACTGGATATGT
CACAAAAGAGGCAGCGGATTTGACAGGATTAAAAGAAGGGACAATAGTTG
TAGGCGGAGGAGGAGATCAAGCAAGTGGAGCAGTAGGGACTGGTACAGTA
AAGAGTGGCATAGTATCAGTTGCACTTGGCACTTCAGGAGTTGTATTTGC
AAGTCAAGACAAATATGTAGTAGATGAAGAAAACAGATTACACTCTTTCT
GTCATGCTAATGGCAAATGGCATGTAATGGGAGTAATGCTCTCAGCAGCC
GCTTGTTTAAAATGGTGGATAGATAACATAATCAACTTTAATGGTTCTTC
TATAACATATGAAAAGCTTTTAGAAGAAGCAGAAAAAGTAACACCAGGAA
GCGGTGGGTTAATATTTCTCCCTTATCTCATGGGGGAAAGGACTCCTCAT
AGTGACCCTTACGCGAGAGGATGCTTTATAGGGCTTAATATGACTCACAA
TAGAGGACATATGACAAGAGCAATACTTGAAGGGGTAGCATTTGGACTTA
GAGACTCGCTTGAGATAATAAAAGAACTTGAGATACCAGTAAATGAAGTT
AGAGTAAGCGGCGGCGGTGCAAAGAGCAAATTGTGGAGGCAAATACTTGC
AGACATTTTTGGTGTCAGAGTAGACATGGTAAATGCGACTGAAGGGCCAG
CTTTTGGCGCAGCAATAATGGCAGCAGTGGGATATGGAATATTTAAAGAT
GTTGAGGAGGCATGTAGTACACTCATCAAAGTGACTGATAGTGTTTACCC
AATAGGAGAAATTGTAAGTAAATATAATGAAATATATCAGATTTATAGAG
GTTTGTATAAGGCCCTTAAAGATAGGTTTGGGGAAATAGCCAGTATAAAT
TAA.
is:
SEQ ID NO. 3
GTGATAAGTAAATCTTTTTATGCGCATCACAGCGCATTTGGCGCTTTCTC
AAGTTTTGTAATCGGTAAATGCGGTAAAGGCGGTGGCGTCGTACTAAATG
ATGTTCGGCCGCCTGAAAACAACGTCTACATTGGATACAAAAGAGATGGT
GTTATAAGCTTGCTGCCATTTATTAAAGATGATACAAAAAATGCTGAAGA
AGAGTTTACAGGAGAAGTCTCTACAAGCAAAAAAGAAAAAAACATAAAAA
TCTTTGGGGAAGATGAGATAGAAAGAGAGTTATGCTGGGCATCAGACACT
TGGACAGCAGGAGACTTCAAATTTTCCATCATCACTCCATTTGGATACGT
AAAAGATCCTTCGGTGATGAATGGAGACGAAAAGAAACTTGCACTGGCAC
CTGTCATATTTGTACAGTTGACAATGGATAATACTGACAGCGATAAGGAT
GCTGAGATGATATTTGGCTTTGAAGGTCCGAAAAGGATATTATCTGAGCT
TACAGATGGAAAATACTTAGGAGGAGTATACGGCAGAAAATACGGTTTTG
CTATCAAAAAAAGCGATGATGTAAGAGAGCTTTCAAGGCTTGATATTTTG
ACATCATGGGCAAATGACAACTATCAAAATCATGGGCTTGGCAGAGCGCC
GTCTTTGATATTTAAAGTGCCGAGAGGGGAGAAAAGGACATATACTGTGG
CATTGGCAACGTATCAAAGCGGCGTCATAACAACAGGAATCGATGCTGAA
TTTTACTACACATCTGTTTTTAAGTCATTGGAAGAAGTATTATCCTTTGG
ACTTGACAATCAAGATTATTACTTAAATTTAGCAAAGGAAAGAGATGAAG
AGCTTAAGAAAAGCGGTTTAAATGAATACAGGCAGTTTTTATTGGCACAT
GCAGCCCACAGTTACTATGCCAGCACGGAGCTTTTAAAGAGAGACGTGGT
ATGCCTCTTTGGGTGGTAAACGAAGGCGAATACATTATGATAAATACATT
TGATTTGACGGTTGATCATGTCTTCTGGGAAATGAGGTTCCATCCTTGGA
CGATTACAAATACATTGGATCTGTACTATGAAAAGTACAGCTACAGGGAT
CAAGCAGGTCTTGCCTTTACGCATGATATGGGTGTCGCAGATGGTTTTTC
TAAAGAAGGCTATTCATCTTACGAGCTTCCAAACCTGACTGGATGTTTTA
GCTACATGACACATGAGGAGCTTTTGAATTGGGTTTTGACAGGTTCTGTC
TATGCAATAAAAATAAATGATAAAGAATGGTTAAAGAAAAACATGGGTGT
ATTCGAAGATTGTTTCGATTCTCTTGTGGCAAGAGATAAAAATAATGATG
GAATAATGGACGTTGACAGTTCAAGGTGTGAGACGGGGTCGGAAATAACG
ACTTACGATAGCCTTGACGAAAGCTTGGGACAGGCGAGAAACAATCTATA
CCTTGGTGTTAAGACATGGGCAGCTTACGTGATGTTGCATGGTTTGTTTA
AAGAAAATGATCTTAGTGAAAAGGCAGAAAAAGCTTTAGAAAAGGCAAGA
CAGGCTGCTAATACTATCGTTGCCAAGTTTGACGAAGAAAATCAGTATAT
ACCTGCAGTATTTGAGAATGGCAACACATCAAGGATAATACCTGCTGTAG
AGGCATTGGTATATCCATATGTTGTAGGATATACTGACTTTGTAAGTGAA
GATGGTGTATTTGGTGGGCTTATAAAAGCCTTAAAGAAGCATGTAATGAC
GATTATGAAGCCTGGTATATGCATAGATGAAGTATCTGGAGGTTGGAAGC
TTTCGTCAACCAGCAAGAACACATGGAATAGTAAAATTTTCTTATGCCAA
TATGTGATAAAAGATGTGCTTAATATAGACTTTGGAGACAAAGAGATTGA
GTGGGACAAAGTACACGCAATGTGGCAACAGGTGTCTTGCAGTGAAGATT
GCGCTACAGATCAGGTAAACAGCGATACAGGTACGCCAAGAGGAAGCCGC
TTGTATCCGAGACTTGTGACAAGTGTATTGTGGATGAAATAG.

### DETAILED DESCRIPTION

Synergistic breakdown of cellulose and hemicellulose is known to make the substrates more accessible by enzymes. Using a single species to ferment both cellulose and hemicellulose rather than a co-culture eliminates the need to control for shifting species populations due to their different optimal growth conditions (e.g., pH), changing availabilities of solid polysaccharides (e.g., glucan, xylan) and soluble sugars (e.g., glucose, xylose) released during polysaccharides degradation. In addition, single-species fermentation will reduce diffusion and nutrient limitations experienced by a co-culture species, resulting in superior process robustness at high solids-loading at large scale.

Consolidated bioprocessing (CBP) of lignocellulosic biomass holds promise to realize economic production of second-generation biofuels/chemicals. However, CBP of the two most abundant biomass components, cellulose and hemicellulose, are currently approached with co-cultures of specialized bacterial species, which compared with a single-species fermentation introduces unnecessary process complexity that may compromise process robustness. Disclosed herein are non-natural organisms (e.g. Clostridium thermocellum) engineered to co-utilize cellulose and hemicellulose. By evolving a previously engineered xylose-utilizing strain in xylose as the only main carbon source, an evolved clonal isolate (KJC19-9) showed improved specific growth rate on xylose by around 3-fold and displayed comparable growth to a minimally engineered strain grown on the bacteria's naturally preferred substrate, cellobiose. To enable xylan utilization, we recombinantly expressed three β-xylosidase enzymes originated from Thermoanaerobacterium saccharolyticum in KJC19-9 and demonstrated growth on xylan with one of the enzymes. This recombinant strain was capable of co-utilizing cellulose and xylan simultaneously, so we integrated the β-xylosidase gene into the KJC19-9 genome, creating KJCBXint strain. Comparing the fermentation of de-lignined, corn stover biomass between strains with and without β-xylosidase, our data showed significantly greater xylan, xylose, and xylobiose consumption in the strain expressing the enzyme (KJCBXint) than the strain without (KJC19-9), and that intermediate xylobiose accumulates in KJC19-9 but not in KJCBXint. This is the first reported C. thermocellum strain capable of degrading and assimilating hemicellulose polysaccharide while retaining its cellulolytic capabilities, and our engineered strain unlocks immense potential of CBP for a better bioeconomy.

Cellulose and hemicellulose, making up structural components of the plant cell wall, are the top two most abundant organic substances on earth. The world is motivated to convert these renewable resources into biofuels and chemicals as it is sustainable, carbon neutral, and provides an opportunity to be carbon negative. However, overcoming plant biomass (i.e., lignocellulose) recalcitrance is key to achieving economic production of bioproducts from these feedstocks and various approaches have been explored. Much of such effort involves chemical or mechanical pre-treating the biomass, engineering and use of cellulase enzymes that hydrolyze the cellulose followed by fermentation of the soluble sugars released from it. One other approach of high interest is consolidated bioprocessing, which relies on the microorganisms to produce cellulolytic enzymes and hence achieve cellulosic biomass hydrolysis, fermentation of the resulting sugars, and generation of the target bioproduct in one integrated process. The latter approach offers a simplified overall bioprocess configuration and reduced processing cost.

Acetivibrio thermocellus most commonly known as Clostridium thermocellum (also known as Rumini Clostridium thermocellum, Hungatei Clostridium thermocellum) is a leading model consolidated bioprocessing organism recognized for its fast solubilization of and growth on cellulosic biomass. This thermophilic and anaerobic bacterium thrives at 55-60° C. and upon growth on cellulosic biomass produces H₂, ethanol, and acetate in larger amounts among other organic compounds. The bacteria produce a suite of hydrolytic enzymes orchestrated in cellulosomes to not only break down cellulose but also hemicellulose. However, it could not naturally grow on xylose or arabinose which are two main sugars making up hemicellulose. Although there has been a large volume of studies aiming at understanding the bacteria's metabolism, improving the production of cellulosic ethanol and other products using engineered C. thermocellum strains, consolidated bioprocessing of both cellulose and hemicellulose is primarily approached with co-cultures of C. thermocellum with bacterial species that utilize hemicellulose and five-carbon (C5) sugars derived from it. Limited progress was made in engineering C. thermocellum to co-utilize cellulose and hemicellulose.

Besides liquid biofuels and chemicals (e.g., ethanol), hydrogen (H₂) is a gaseous product naturally released in copious amounts by C. thermocellum during dark fermentation of the biomass and the release of H₂ is critical for bacterial fitness. Considering the forecasted global energy matrix, bio-H₂ has emerged to play a crucial role in mitigating greenhouse gas when coupled with efficient CO₂ removal and storage. However, a major roadblock for fermentative H₂ production is incomplete feedstock deconstruction, utilization, and conversion, which leads to low H₂ yield. This highlights the economic impact of efficient co-utilization of both cellulose and hemicellulose for downstream production of H₂ and a wide range of target bioproducts.

Taking steps toward co-utilizing cellulose and hemicellulose, our earlier effort integrated the xylose isomerase and xylulose kinase genes (i.e., xylAB) into C. thermocellum genome. Although the functional expression of the xylose-catabolizing pathway conferred simultaneous co-utilization of xylose with cellulose, cellobiose, and glucose, respectively, growth on xylose was substantially slower than its optimal growth on the bacteria's preferred substrates (e.g., cellobiose, Avicel). Furthermore, incorporation of xylAB genes alone did not confer growth on xylan or xylo-oligomers as the main carbon source.

Accordingly disclosed herein is a non-naturally occurring C. thermocellum engineered for co-utilization of hemicellulose and cellulose through both adaptive laboratory evolution (ALE) and rational strain engineering.

Adaptive laboratory evolution improved C. thermocellum growth on xylose. The xylose isomerase and xylulose kinase encoded by xylAB genes have been recombinantly expressed in C. thermocellum and enabled this cellulose-utilizing bacterium to also grow on xylose. However, the engineered strain displayed compromised growth rates and reached lower maximal culture density on xylose compared to its growth on cellobiose, a soluble and preferred 6-carbon model substrate for the bacteria. To test the feasibility and effectiveness of adaptive laboratory evolution (ALE) on improving the bacteria's growth on xylose, we serially sub-cultured C. thermocellum transformants bearing the xylAB expression on a plasmid for nearly 100 generations (20 passages). An evolved clonal isolate was shown to grow on 5 g/L xylose with reduced initial growth lag, which provided the proof of principle that the engineered strain had the genetic capacity to grow well on xylose.

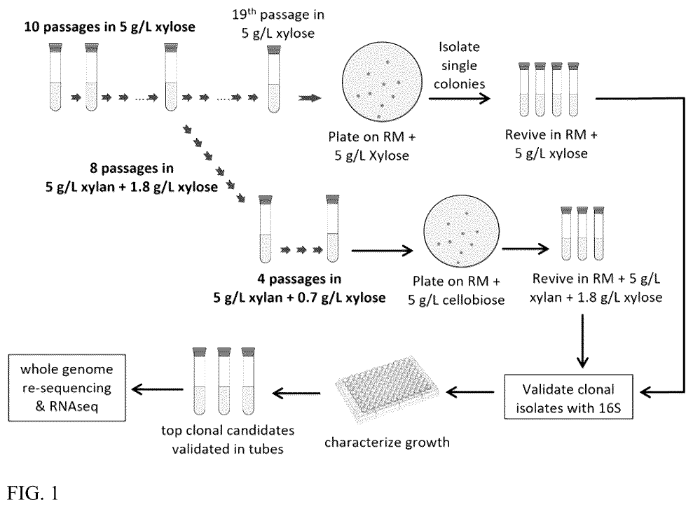

Subsequent ALE started with the integrated xylAB genes in the genome (KJC335). This parental strain was selected with 5 g/L xylose as the only carbon source over 19 passages and single colonies were isolated on a rich media agar with xylose (FIG. 1). There has been previous evidence for xylo-oligomer utilization in the presence of xylose monomers by KJC335. Therefore, an alternate ALE approach was taken to isolate strains with enhanced xylose and xylan utilization. After the 10th passage in xylose, the culture was diverted to 8 passages in 5 g/L xylan with 1.8 g/L xylose followed by an additional 4 passages in 5 g/L xylan with 0.7 g/L xylose, and plating on cellobiose to isolate colonies. After characterizing the growth of isolates in a 96-well plate, two of the best-growing isolates from each evolutionary pathway were selected, which were, KJC19-1 and KJC19-9, from the ALE with xylose as the only main carbon source, and KJCXn4 and KJCXn6, from the alternative ALE selected with xylose and xylan.

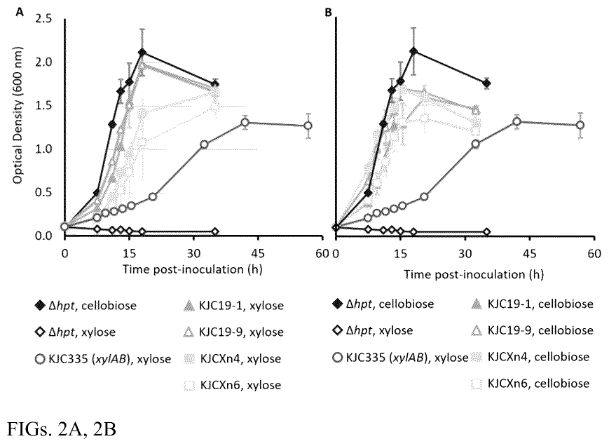

The four isolates grew better than the unevolved strain on 5 g/L xylose, but KJC19-1 and KJC19-9 displayed even higher growth rates than either KJCXn4 or KJCXn6, nearly reaching the rate of the minimally engineered strain, Δhpt, on 5 g/L cellobiose (FIG. 2A). This suggests that the addition of xylan as a selective pressure was not as effective in improving the bacteria's growth on xylose. Additionally, KJCXn4 and KJCXn6 did not seem to have enhanced ability to grow on xylan based on the low optical densities of the cultures when grown in 5 g/L xylan, 1.8 g/L xylose, or the combination of two. This suggests that C. thermocellum does not have the genetic capacity to fully degrade xylan and this remains a rate-limiting step. Lastly, when tested for how well these strains retained their innate ability to grow on cellobiose, KJC19-9 and KJCXn4 displayed similar growth rates with Δhpt but reached a lower maximal culture density (FIG. 2B).

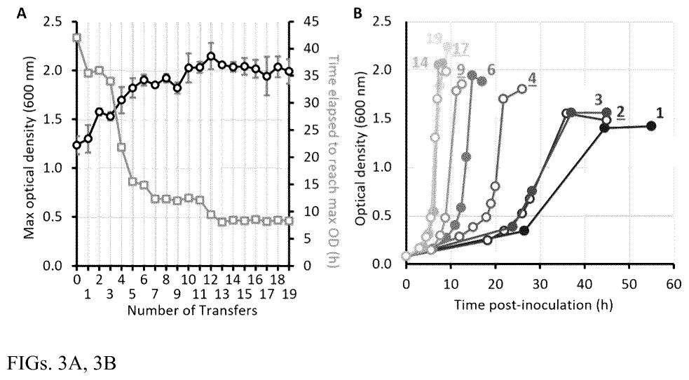

By examining the xylose-derived ALE cultures more closely, the data showed that the maximal optical density (OD₆₀₀) of KJC19-9 increased significantly from 1.2 by the unevolved parental strain to 2.0 by the evolved culture, and the time required to reach maximum culture density dropped from 42 to 8.3 hours (FIGS. 3A, 3B). Substantial growth improvements on xylose were seen during the first 6 passages and minimal to no improvements in growth rate or max OD were observed after transfer 13 or 14 (FIGS. 3A, 3B). ALE-produced strains are well suited for growth on xylose with minimal loss in capacity for the bacteria's native carbon source.

The evolved mutants descending from plasmid-based and genome-integrated expression of xylAB genes, KJC19-9, KJCXn4, and their respective ancestral strains were sent for genome re-sequencing. Comparing the genome sequence output amongst KJC335, KJC19-9, and KJCXn4 against the DSM 1313 published genome, a total of 46, 56, and 58 mutations were observed, respectively. Of these mutations, 3 were unique to KJC335. 14 additional unique mutations were observed in KJC19-9 and 15 in KJCXn4. 10 of these unique mutations were common between KJC19-9 and Xn4 (Table 1), which suggested shared adaptive events between the two strains possibly during the first 10 passages in xylose (5 g/L) before their ALE courses diverged. Additional characterizations of the genetic changes will be detailed in a later study with transcriptomic data to understand the changes in the different strains at the transcriptomic level.

TABLE 1
Summary of Strains and Plasmids.

| Plasmid & | Parent Source | Relevant Descriptions of | Accession | |
|---|---|---|---|---|
| Strain ID | & Host Strain | the Strain and Plasmid | number | Reference |
| pKJC84-1 | | empty shuttle vector for E. | | Ref (50) |
| | | coli and C. thermocellum | | |
| pKJC150-2 | pKJC84-1 | plasmid-based expression of a | AFK86459.1 | Generated in |
| | | β-xylosidase (Tsac_1452, xylB), | | this study |
| | | matching actual genome sequence | | |
| pKJC152-1-A | pKJC84-1 | plasmid-based expression of a | AFK86459.1 | Generated in |
| | | β-xylosidase (Tsac_1452, xylB), | | this study |
| | | mutated to match published | | |
| | | genome sequence | | |
| pKJC155-38 | pKJC84-1 | plasmid-based expression of a | AFK86458.1 | Generated in |
| | | β-xylosidase (Tsac_1451, XylD), | | this study |
| pKJC184-3 | pKJC84-1 | plasmid-based expression of a | ABM68042.1 | Generated in |
| | | β-xylosidase (Tsac_0939, XylC) | | this study |
| pLH06 | pKJC84-1 | plasmid-based expression of | FJ591151.1 | Generated in |
| | | T. ethanolicus XylAB | | this study |
| Thermoanaerobacterium | DSM 8691 | Aquired from DSMZ | | |
| saccharolyticum | | | | |
| KJC315 | DSM 1313 | Δhpt | | Ref (29) |
| KJC335 | KJC315 | Δhpt::PGapDH-xylA-xylB (unevolved) | | Ref (50) |
| KJC19-1 | KJC335 | an evolved, clonal isolate | | Generated in |
| | | (selected with xylose) | | this study |
| KJC19-9 | KJC335 | an evolved, clonal isolate | | Generated in |
| | | (selected with xylose) | | this study |
| KJCXn4 | KJC335 | an evolved, clonal isolate | | Generated in |
| | | (selected with xylan plus xylose) | | this study |
| KJCXn6 | KJC335 | an evolved, clonal isolate | | Generated in |
| | | (selected with xylan plus xylose) | | this study |
| LH012 | DSM 1313 | Δhpt/pLH06 (unevolved) | | Generated in |
| | | | | this study |
| LH33202 | LH012 | Δhpt/pLH06 (evolved in xylose) | | Generated in |
| | | | | this study |
| KJC391 | KJC19-9 | KJC19-9/pKJC84-1 | | Generated in |
| | | | | this study |
| KJC392 | KJC19-9 | KJC19-9/pKJC150-2 | | Generated in |
| | | | | this study |
| KJC393 | KJC19-9 | KJC19-9/pKJC152-1-A | | Generated in |
| | | | | this study |
| KJCBX | KJC19-9 | KJC19-9/pKJC155-38 | | Generated in |
| | | | | this study |
| KJC395 | KJC19-9 | KJC19-9/pKJC184-3 | | Generated in |
| | | | | this study |
| KJCBXint | KJC19-9 | integration of T. sacc xylD in | | Generated in |
| | | the KJC19-9 genome as the last | | this study |
| | | gene of the “Δhpt::PGapDH-xylA- | | |
| | | xylB” synthetic operon | | |

Engineering the evolved mutant to co-utilize xylan and cellulose. Despite efforts to evolve KJC335 on 5 g/L xylan supplemented with xylose, KJCXn4 and KJCXn6 were unable to satisfactorily grow on xylan as the sole carbon source. The xylan backbone is composed of β-1,4-linked xylose and is the major component of hemicellulose. The backbone often contains side groups including arabinose, glucuronic acid, acetic acid, ferulic acid, and p-coumaric acid. In addition to the enzymes required to remove these sidechains, endo-β-1,4-xylanase and β-xylosidase are required for the complete depolymerization of the xylan backbone. Endo-β-1,4-xylanase cleaves xylan internally and releases xylo-oligomers, whereas β-xylosidase has exo-activity and further degrades the xylo-oligomers into monomeric xylose from the non-reducing end.

C. thermocellum possesses endo-β-1,4-xylanases and many have been characterized. Low levels of β-xylosidase activity against synthetic substrates have been detected in cell extracts of C. thermocellum strain ISOII, however, no enzyme has been identified. Additionally, the construction of KJC335, C. thermocellum strain DSM 1313 with the xylAB pathway depicted that the bacteria had some capacity to consume xylooligomers with degrees of polymerization of 2-7 in length if xylose monomer was also provided. Evidence for xylo-oligomer utilization included decreased xylobiose and xylotriose concentration over time and doubled product formation (biomass, acetate, and ethanol) in the combined 3.5 g/L xylo-oligomer with 1.5 g/L xylose fermentation compared to growth on 1.5 g/L xylose alone. The doubled product formation is low when one considers the cells having about 3.5 times more carbon source in the xylo-oligomer fermentation, suggesting xylo-oligomer deconstruction is limiting. Some xylose production is possible by various C. thermocellum endo-β-1,4-xylanases, and it's possible that these enzymes were upregulated in the presence of xylose. Additionally, some of the disappearances in xylobiose and xylotriose could be due to unidentified transglycosylase activity. In the same paper, it was also found that based on isotope labeling, cells produced 30% of their ethanol from xylan when they were grown with 2.5 g/L ¹³C-glucose and 2.5 g/L xylan (¹²C) from beechwood. However, the extent of xylan utilization was not known considering that glucose may be present in the xylan from beechwood and that the xylan utilization may be a result of xylose release from endo-xylanase activities to a lesser extent. Though there is evidence for some xylan to xylose deconstruction, C. thermocellum DSM 1313 does not produce a true β-xylosidase. We hypothesized introduction of a β-xylosidase encoding gene would give KJC19-9 the ability to efficiently utilize xylan as a carbon source.

We chose to explore the three characterized β-xylosidase enzymes from Thermoanaerobacterium saccharolyticum, which is a thermophilic anaerobe that can grow on xylan as a sole carbon source.

XylB (Tsac_1452) was the first β-xylosidase from T. saccharolyticum strain B6A-RI studied using recombinantly expressed XylB from E. coli, and the homolog in T. saccharolyticum JW/SL YS485 was characterized shortly thereafter. XylB has an optimum activity at pH 5.5-6.0 and 65-70° C. XylB produced xylose from xylobiose, xylotriose, and xylopentose. The enzyme exhibited transglycosylase activity, which was demonstrated by the appearance of xylotriose when xylobiose was used as the substrate. The deletion of xylB didn't have a clear phenotype when it was grown with xylan as the carbon source. The strain did not accumulate xylan and xylooligomers, nor did it have lower ethanol production compared to the wild-type T. saccharolyticum strain M0355.

XylC (Tsac_0939) was purified from crude T. saccharolyticum JW/SL-YS485 extracts and recombinantly expressed in E. coli. XylC was shown to hydrolyze xylobiose and xylotriose with an optimum activity at pH 6 and 65° C. XylC has a Km value of 28 mM and a kcat of 189,292. Others have found XylC can perform both hydrolysis and transglycosylation reactions. In the presence of various alcohols and pNP-xyloside, XylC produced alkyl alcohols.

XylD (Tsac_1451) was originally characterized as a xylose isomerase and later recharacterized as a β-xylosidase. XylD has a Km value of 0.68 and a kcat of 5,075. Unlike XylB, others found that the deletion of xylD decreased ethanol production and led to an accumulation of short-chain xylo-oligomers when cells were grown with xylan as the carbon source.

The respective xylB, xylC, and xylD genes were amplified from purified T. saccharolyticum JW/SL-YS485 genomic DNA and expressed from the gapDH promoter on a plasmid in KJC19-9. The xylB gene sequence did not align with the published genome sequence and a second construct was synthesized to match; both plasmids produced the same result.

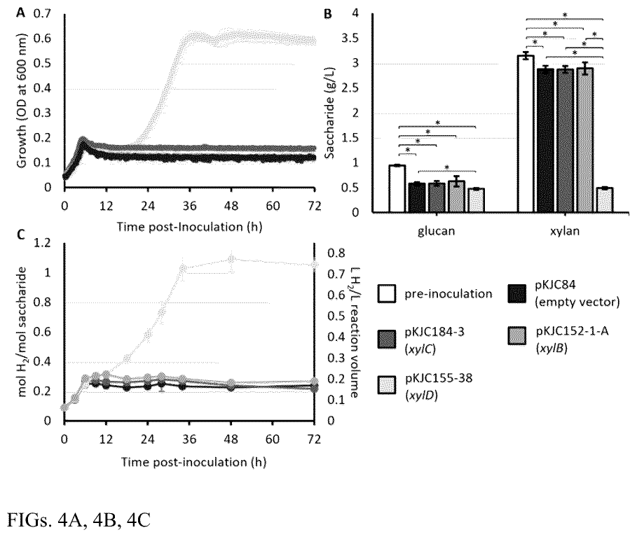

KJC19-9 carrying pKJC84 (empty vector), pKJC152-1-A (xylB), pKJC184-3 (xylC), and pKJC155-38 (xylD) were grown on 5 g/L xylan from corn core. Our results showed that empty vector and expression of xylB or xylC in the KJC19-9 strain conferred similar but minimal growth with xylan (FIG. 4A). However, KJC19-9 expressing xylD was able to grow well with xylan as the sole carbon source (FIG. 4A). Based on the analysis of the remaining acid-hydrolyzed sugars, expression of xylD enabled 84% utilization of the xylan, whereas the other strains only used 8 to 9% (FIG. 4B) which suggested consumption of xylose released in small quantities from degradation of xylan by endo-xylanase(s) native to C. thermocellum The xylD expressing strain was able to consume about 10% more glucan than KJC19-9 with empty vector, suggesting that more complete degradation of xylan gave the bacteria access to utilize these residues from the polysaccharide. KJC19-9 with xylD produced 4 to 5 times more hydrogen than the other strains and was the only strain able to produce substantial or detectable amounts of lactate, formate, and ethanol due to more complete xylan deconstruction and consumption of the xylose monomers (FIG. 4C).

Without being limited by theory, it is not clear why T. saccharolyticum is equipped with three different β-xylosidases, but their functions may be complementary for xylan deconstruction. Of the three β-xylosidases tested, XylD is the only one that improved xylan utilization. Based on our data, XylD is likely responsible for hydrolyzing xylobiose and xylotriose into monomers, whereas XylB and XylC might have other responsibilities relating to their transglycosylation activity that has been detected. The monomeric xylose in the xylC-expressing culture accumulates to a level over two-fold higher than the others. In an embodiment, expressing these β-xylosidase enzymes together in C. thermocellum strains would be useful for optimizing co-utilization of cellulose and hemicellulose.

Simultaneous co-fermentation of xylan and cellulose. Co-fermentation of cellulose and hemicellulose is desired as a synergistic deconstruction of these lignocellulosic components enables efficient enzyme access to their respective substrates. In addition, hydrolysis of both cellulose and xylan simultaneously without carbon catabolite repression (CCR) may further reduce fermentation costs. However, intermediates such as xylo-oligomers released from hydrolysis have been shown to be strong inhibitors of cellulose hydrolysis. We tested whether cellulose and xylan can be hydrolyzed simultaneously, and whether cross-inhibition may be apparent amongst the soluble sugars including monomeric and oligomeric six- and five-carbon sugars.

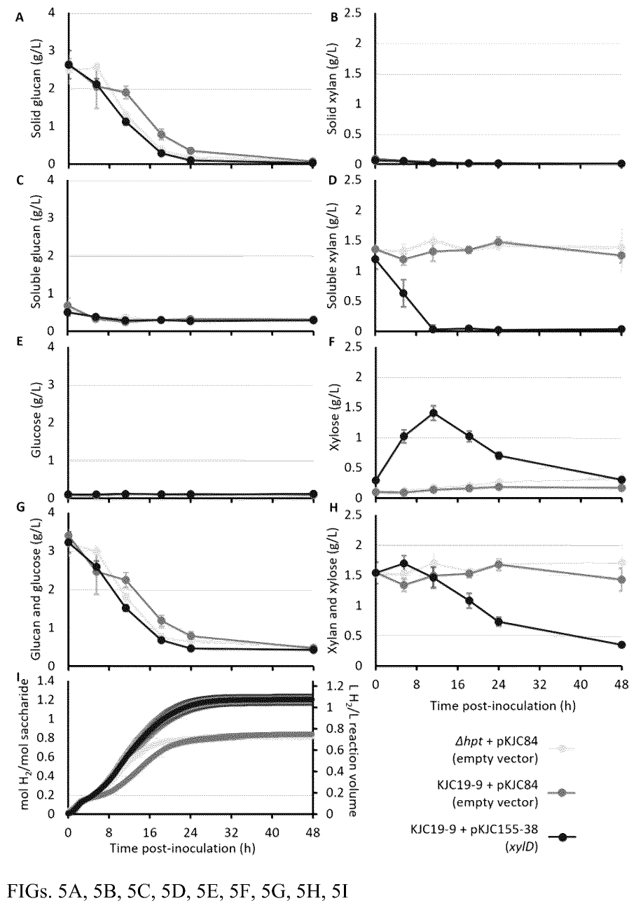

We compared Δhpt with pKJC84 (empty vector), KJC19-9 with pKJC84, and KJC19-9 with pKJC155-38 (xylD) for their ability to co-ferment 2.5 g/L Avicel with 2.5 g/L xylan from corn core and produce hydrogen. All three strains degraded the cellulose (solid glucan) at a similar rate (FIG. 5A), and the cellodextrins were quickly consumed by C. thermocellum, indicated by their lack of accumulation in the soluble sugars fraction (FIGS. 5C, 5E). By the end of fermentation, 83-86% of the glucan was consumed by all three strains (FIG. 5G). Xylan from the corn core is completely soluble and therefore minimal amounts were detected in the solids fraction (FIG. 5B). The soluble xylan was rapidly degraded by KJC19-9 with pKJC155-38 and nearly completed by 11.25 hours (FIG. 5D). Simultaneous accumulation of xylose was observed over the first 11.25 hours in the fermentation by KJC19-9 bearing pKJC155-38. This was followed by a decrease in xylose over the next 37 hours due to the assimilation of the xylose monomer by the bacteria (FIG. 5F). There was little to no conversion of xylan or xylo-oligomer to xylose detected by Δhpt or KJC19-9 with empty vectors (FIGS. 5D, F), which suggests again that C. thermocellum does not possess a native β-xylosidase for conversion of xylobiose or xylotriose into xylose. 111% and 92% of xylan remained for Δhpt and KJC19-9 with empty vectors, respectively (FIG. 5H). KJC19-9 with pKJC155-38 had only 21% xylan remaining at the end of the 48-hour fermentation.

The expression of T. saccharolyticum xylD in KJC19-9 (KJC19-9/pKJC155-38) successfully utilized the Avicel and xylan from corn core simultaneously, indicated by the downward slope of xylan from sampling points between 5.5 and 48 hours (FIGS. 5G, 5H). However, the larger negative slopes of glucan compared to xylan, max 0.17 g/L/h vs 0.05 g/L/h, respectively, suggests more efficient degradation and/or assimilation of Avicel compared to xylan. In addition, the ability to complete the xylan degradation process allowed the xylD expressing strain to generate 46-50% more hydrogen compared to Δhpt or KJC19-9 with empty vectors over 48 hours (FIG. 5I), demonstrating the feasibility to increase a target product yield from cellulose and xylan co-utilization.

Co-utilization of pentose (e.g., xylose) and hexose (e.g., glucose) sugars without carbon catabolite repression (CCR) reduces fermentation time and has been earnestly sought after. The co-fermentation of xylose and cellobiose without the CCR has been reported in engineered model mesophilic organisms including yeast and E. coli. However, neither of these organisms is engineered to be cellulolytic or could directly utilize complex, polysaccharides such as cellulose or xylan. Success in engineering yeast to exhibit cellulolytic activities is in strains that cannot ferment pentose sugar. Among thermophilic bacteria, CCR mechanisms in T. saccharolyticum has been studied and the deregulation resulted in altered order of sugar utilization, but this bacterium is not cellulolytic. C. bescii can do it but is slow. We have demonstrated a unique engineered strain that simultaneously utilizes refined crystalline cellulose and xylan amid moderate xylose accumulation followed by its consumption.

The accumulation of xylose in the effluent over the first 11.25 hours by KJC19-9 with pKJC155-38 might suggest that the β-xylosidase is secreted. However, this is unclear considering the protein does not have a strongly only has a 33% probability predicted of having a SPII secretion signal according to SignalP-5.0. In comparison, XylB and XylC predicted probabilities for a SPII sequence are less than 0.2%. The activities of two of the three β-xylosidase enzymes we tested, XylD (Tsac_1451) and XylC (Tsac_0939) were detected from crude cell extracts. We tested the C. thermocellum cell pellet extract and supernatant for XylD activity against xylobiose and detected it in both places, but more activity and protein were detected in the cell pellet. Currie et al. previously performed proteomics on T. saccharolyticum and identified XylD in both cell pellet and supernatant fractions as well. It's possible that the secretion signal detected is real but relatively weak, and the protein has a dual localization. Without being limited by theory, the extracellular localization of XylD could help explain why XylD, but not XylB or XylC, allows for growth on xylan. We were unable to determine which one of the two populations of the protein was responsible for the utilization of xylan, but based on the accumulation of xylose in the effluent at 11 hours we hypothesize it's the extracellular fraction (FIG. 5F).

In an embodiment, an experiment using 19-9 pkjc84-xylD with globomycin on xylan vs cb is contemplated. In another embodiment, an experiment using 19-9 pkjc85-xyldD (ΔN-term) on xylan is contemplated. In an embodiment, an experiment using 19-pkjc85-xylD (ΔN-term) intracellular and extracellular activity is further contemplated.

Utilization of lignocellulosic biomass at higher loadings. After confirming the co-utilization of cellulose and xylan by the KJC19-9 with pKJC155-38 (xylD), we decided to integrate the xylD gene to improve the stability of the co-utilizing strain. This is advantageous for two main reasons; the strain no longer requires antibiotic selection to retain the β-xylosidase activity, and the removal of plasmid and antibiotic selection allows for further engineering of the strain. The xylD gene was integrated downstream of xylA and xylB with a ribosome-binding site in frame, creating a new strain, KJCBXint, so all three genes were expressed by the same GAPDH promoter at the deleted hpt gene locus. The correct integration of xylD at this region was confirmed by PCR and sequencing.

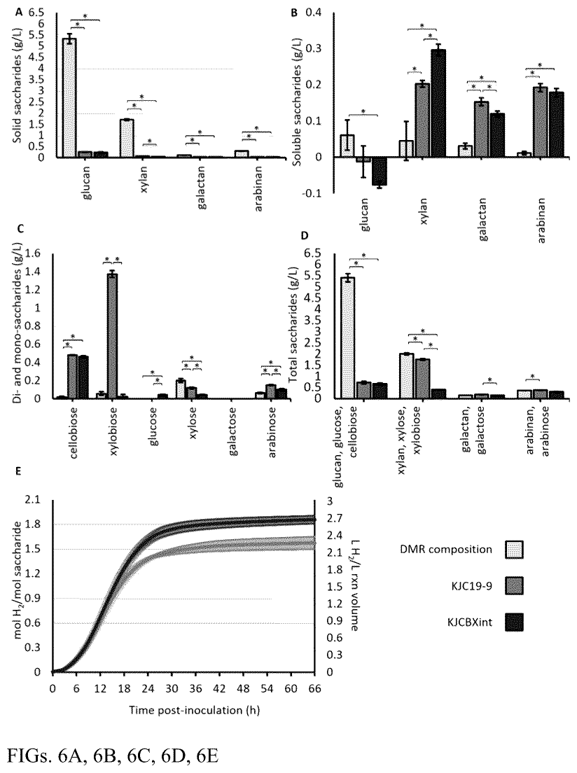

KJCBXint was compared against the xylose-evolved KJC19-9 strain to consume lignocellulosic biomass and produce hydrogen gas. Bioreactors were loaded with deacetylated and mechanically refined corn stover (DMR) at near 5 g/L as cellulose (31.25 g/L DMR wet weight). Based on our data and the composition of DMR at this loading, most of the sugars are bound up in complex, insoluble polysaccharides (FIGS. 6A, B, C, D). At the end of the fermentation, both KJC19-9 and KJCBXint had solubilized similar amounts (95%) of solid polysaccharides (FIG. 6A). Only small amounts (<0.3 g/L) of soluble complex saccharides remained for KJC19-9 and KJCBXint, but KJCBXint had about 0.1 g/L more xylan compared to KJC19-9 (FIG. 6B). The most dramatic difference between KCJ19-9 and KJCBXint is demonstrated in the soluble di- and monosaccharides that remained at the end of fermentation (FIG. 6C). KJC19-9 accumulated 1.4 g/L xylobiose compared to only 0.2 g/L xylobiose by KJCBXint, suggesting that wild-type C. thermocellum can degrade xylan polysaccharide to short xylooligomers, but does not have a dedicated β-xylosidase like XylD to degrade the xylo-oligomers into xylose. The 6-carbon sugar utilization was similar between both strains, but KJC19-9 only consumed 12% of 5-carbon sugars compared to 80% by KJCBXint (FIG. 6D). This additional utilization of the DMR increased the total hydrogen produced by 20% (FIG. 6E).

KJCBXint as disclosed in embodiment presented herein, is the first reported C. thermocellum strain that has the ability to degrade and assimilate the most abundant hemicellulose polysaccharide, xylan, while retaining its cellulolytic capabilities. Implicated by the increased hydrogen gas production through greater usage of biomass components, i.e., cellulose and hemicellulose, KJCBXint is a promising strain for increased downstream product formation (e.g., ethanol, isobutanol, etc.) from a feedstock made of both cellulose and xylan. In addition, KJCBXint serves as a platform strain to express enzymes to cleave specific bonds which link sugars to the hemicellulose backbones. At high solids loading, which is required to make consolidated bioprocessing economically viable, xylooligomer accumulation inhibits solubilization of biomass and growth of C. thermocellum. Using non-naturally occurring C. thermocellum disclosed herein helps to overcome this barrier.

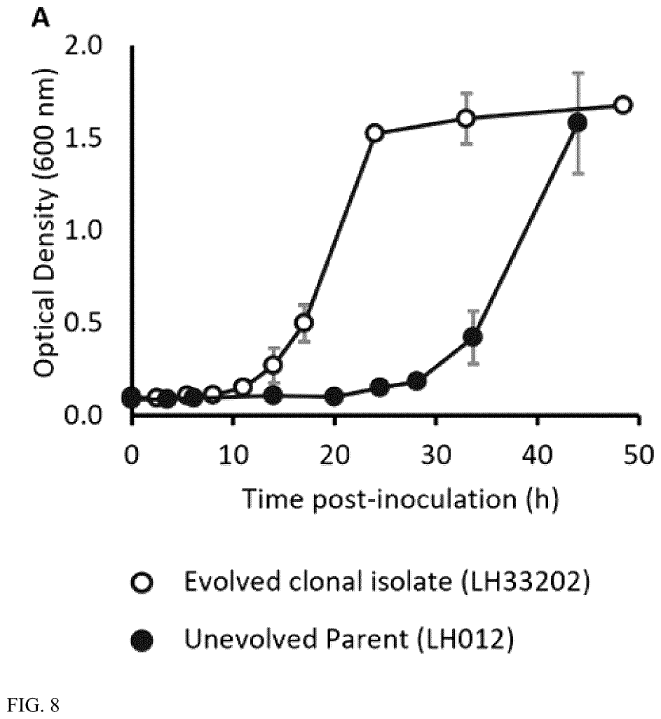

To improve the growth of the bacteria on xylose, we conducted adaptive laboratory evolution (ALE) by serially sub-culturing C. thermocellum transformants bearing the xylAB expression on a plasmid (LH012) in 5 g/L xylose as the main carbon source. After evolving LH012 for nearly 100 generations (20 passages), a clonal isolate (LH33202) was shown to grow on 5 g/L xylose with reduced initial growth lag (FIG. 8), which provided the proof of principle that the engineered strain had the genetic capacity to grow well on xylose, and ALE was indeed feasible and effective in improving growth on xylose.

Whole-genome re-sequencing of the evolved mutants. LH33302, KJC19-9, KJCXn4, and their respective ancestral strains were sent for genome re-sequencing to understand the genetic changes that enabled the growth improvements on xylose. By mapping all re-sequenced genomes against the published DSM 1313 genome sequence, 42 variants were observed in LH012, and 48 variants were found in LH33202. All 42 variants in LH 012 were common mutations to both LH012 and LH33202, leaving 6 unique mutations to LH33202. These 6 mutations were presumably acquired during ALE for the growth improvement observed. Comparing the genome sequence output amongst KJC335, KJC19-9, and KJCXn4 against the DSM 1313 published genome, a total of 46, 56, and 58 mutations were observed, respectively. Of these mutations, three were unique to KJC335. 14 additional unique mutations were observed in KJC19-9 and 15 in KJCXn4. 10 of these unique mutations were common between KJC19-9 and Xn4 which suggested shared adaptive events between the two strains possibly during the first 10 passages in xylose (5 g/L) before their ALE courses diverged.

None of the unique variants in LH 012 were in common with the unique variants in KJC335, KJC19-9, and KJCXn4, providing evidence for different evolutionary pathways to achieve fast growth on xylose between the plasmid vs. genomic expression of the xylAB genes. This may also suggest that additional and distinct evolutionary pathways exist and can confer the phenotype of improved xylose utilization. Although the synthetic operons to express the xylAB genes were designed to be identical between the plasmid and the genomic copy, maintenance of the extra copies of the xylAB genes on a plasmid may exert a metabolic burden on the cells that the integrated xylAB genes in a single copy do not, which can contribute to the distinct evolutionary courses. With only a copy of the xylAB genes and possibly limiting XylAB proteins in the cells, the lineage may have experienced more stringent selective pressure and consequently led to a greater number of synergistic mutations to adapt to growth on xylose. In addition, we also observed that growth improvements on xylose were primarily shortened initial lag phase in 33202 while the growth rates were not substantially higher in the descending cultures (FIG. 8). On the other hand, improvement toward higher growth rates and reduced lag phase were both seen in strains having genomic expression of the xylAB genes. These phenotypes were consistent with and could be consequences of the distinct ALE courses.

Waste biomass rich in cellulose is typically recalcitrant and not easily digested by the microbial communities found in anaerobic digestion. The recalcitrance of cellulosic materials causes issues in many existing waste treatment and valorization technologies (e.g., biogas anaerobic digesters, wastewater secondary treatment) and adds to the retention time, therefore increasing the cost and space required, of these technologies. Our technology offers a solution to efficiently degrade cellulose in waste streams and valorize it for H₂ production.

Technologies which can convert wastes streams high in chemical oxygen demand (COD) often involve the use of electrodes and are more commonly suitable for waste/aqueous streams. Our technology can directly utilize solid, organic, biomass and provides a solution to valorize these wastes for biohydrogen production.

Based on technoeconomic analysis of our invention disclosed herein, for every kilogram of hydrogen produced, the fermentative approaches reduce the electricity consumption by over 55% compared with PEM electrolyzers. Alternative technologies such as gasification of biomass is one other solution to address solid waste but is in general energy intensive and less flexible in scales (e.g., scale up, scale down) compared to our fermentative technology.

Fermentative biohydrogen production also offers a remarkable decarbonizing potential not found in clean hydrogen production via electrolyzers by splitting water molecules.

Disclosed herein are methods and compositions of matter useful for the production of biohydrogen from a broad range of biomass as feedstocks, including Sargassum species (seaweed), mechanically separated fibrous material from cow manure (dairy waste), hard and soft almond shells, coffee chaff, and mint slug (mint plants after oil extraction), and our current standard feedstock, corn stover. This process involves feeding individual feedstocks to an efficient cellulose-degrading bacterium (i.e., Clostridium thermocellum) that ferments the waste biomass and produces biohydrogen as a fermentation product. Whether the biomass is lignocellulosic or algal, it is composed largely of photosynthetically fixed CO₂ forming the polysaccharides and/or carbohydrates that make up the biomass. Fermentation and consumption of these polysaccharides by C. thermocellum generates H₂, and the CO₂co-produced was reasoned to have initially come from atmospheric CO₂. Thus, based on carbon balance, we have demonstrated an overall carbon-neutral bioH₂ production technology.

More specifically, we demonstrated growth and H₂ production from an engineered C. thermocellum DSM 1313-derived strain, “KJCBXint-evo”, on these feedstocks. “KJCBXint-evo” is an engineered C. thermocellum strain first engineered with a xylose-catabolizing pathway in the genome, then evolved in xylose as a selective pressure to achieve improved growth in xylose, then further integrated with a β-xylosidase encoding gene in the genome by design at a specific locus, and then once again further evolved in xylan from corn core (semi-refined, containing 71% xylan and 13.5% glucan by weight) as the main carbon source to improve its utilization of xylan, described below. The strain generated up to the incorporation of the β-xylosidase encoding gene but before adaptive laboratory evolution driven by growth in xylan is disclosed herein.

Briefly, the evolution of this previously reported strain was done by serially transferring the original strain into two tubes with 10 mL rich medium containing 5 g/L of the corn core xylan. The growth of both tubes was tracked over time, and the tube with the highest and/or fastest growth was transferred to two new tubes. This transfer protocol was followed for twenty transfers, and clonal subpopulations (“clones”) of bacteria present in the top-growing culture of the twentieth transfer were isolated and assessed for growth on the xylan. The top-growing clone was further validated with biological repeats and used to screen various biomass types for viability for biohydrogen production.

For all biomass types except Sargassum species, the biomass was sterilized (mint slug and dairy waste were initially autoclaved for safe handling, and were later autoclaved for the second time as the experiments were setup), dried (air-dried or lyophilized), milled into 0.5 mm particles, and loaded at 5 g/L as cellulose into 5 mL fermentations in tubes. All feedstocks were autoclaved prior to fermentation. For the fermentation of the cellulose-forming macroalgae (seaweed) Sargassum species, we used a commercially available dried powder of Sargassum as the feedstock in this experiment, loaded at 20 g/L total biomass into 5 mL fermentations in tubes. The fermentation was run for 15 days for all biomass types, and cumulative hydrogen production was tracked by gas chromatography.

Sargassum was purchased commercially as a dried powder and its composition has not been characterized at the time of this filing, but it is expected to contain cellulose (glucan) and other polysaccharides. The fermentation of Sargassum led to hydrogen generation of 0.44 L H₂ per L reaction volume during the fermentation.

We tested fibrous material from cow manure. This material is a byproduct of dairy production, as the fiber is extracted from cow manure before the remainder of the manure is channeled to an anaerobic digester for methane production. The batch we sourced contained 24% glucan and 14.8% xylan by dry weight. This fiber is typically sundried and reused as bedding for cows but until now has been considered an extremely low-value industrial byproduct with little valorization potential. However, C. thermocellum efficiently valorized this waste and produced 0.51 L H₂/L reaction from the material.

Hard and soft almond shells are typically sold for use as bedding in cattle/dairy operations and may be an economical feedstock source for valorization, with a projected 1.8 billion tons of almond shells being produced in California in 2023. The batch of hard almond shells we sourced contained 23.2% glucan and 19.7% xylan by dry weight, and the batch of soft almond shells we sourced contained 21.1% glucan and 17% xylan by dry weight. Hard almond shells led to the production of 0.36 L H₂/L reaction, and soft almond shells led to the production of 0.46 L H₂/L reaction.

Mint “slug”, or spent mint plant, is the residual biomass after steam extraction of mint oil. We sourced the mint slug from a farming community in central Washington State. Our sample could be a mix of peppermint, spearmint, and more. The batch we sourced contained 17.3% glucan and 5.4% xylan by dry weight. Fermentation of mint slug led to the production of 0.47 L H₂/L reaction.

Coffee chaffs are the dried skin on a coffee bean, or the husk, which comes off during the roasting process. The batch we sourced contained 19.7% glucan and 5.6% xylan by dry weight. Fermentation of coffee chaff led to the production of 0.37 L H₂/L reaction.

Corn stover sourced from Idaho contained 35.5% glucan and 20.8% xylan by dry weight. The corn stover led to the production of 0.95 L H₂/L reaction. Corn stover is a model substrate for our R&D efforts and was used as a reference feedstock to compare yields by other novel biomass types. This hydrogen yield from corn stover obtained with the adaptively evolved strain, “KJCBXint-evo”, is roughly a 40% increase in hydrogen yield relative to a refined cellulose (Avicel) control versus the unevolved strain which was the subject of a previous ROI. In other words, the adaptive evolution of this strain reported here boosted hydrogen yield from milled corn stover by 40% in a preliminary comparison of two experimental datasets. Future research efforts will determine the exact improvement in hydrogen yield from corn stover resulting from the adaptive evolution of the strain.

What was the problem solved by the invention, or what was the need for the invention?

Biohydrogen (bio-H₂) production from organic waste streams is recognized as a viable renewable alternative to H₂ produced from fossil fuels. This biological pathway enabled by microbial fermentation of biogenic materials as a feedstock is a carbon-neutral technology for hydrogen production, but it also provides a unique decarbonization potential that is lacking in H₂ production using water-splitting technologies via an electrolyzer. More specifically, through biomass fermentation using our technology, whether the starting feedstock be algal biomass or lignocellulosic plant biomass, both hydrogen and CO₂ are co-produced in near 1:1 ratio in relatively high purity. Such relatively concentrated stream of CO₂ released from the fermentor can be sequestered by point-source carbon capture and sequestration rather than direct air carbon capture which is more energy intensive. In other words, after the fermentation, the atmospheric carbon dioxide, upon being fixed into algal or lignocellulosic biomass and fermented, was traded with a concentrated source of CO₂. This concentrated CO₂ stream has the potential to be utilized for carbon capture and storage (CCS) technologies to make the technology carbon-negative.

Several waste biomass streams including coffee chaff, diary waste, and mint slug shown to be suitable feedstocks with our technology are low-value byproducts and are commonly landfilled or otherwise disposed. A long-term vision of this technology is to build a nationwide green H₂ infrastructure and create a network of decentralized, local H₂ producers to empower clean H₂ consumers in communities across the country. This decentralized H₂ economy reduces the life-cycle greenhouse gas emissions from transporting H₂ from the source of its production to sites where it is utilized. A decentralized H₂/power platform also provides energy security as a grid system can be interrupted/damaged due to natural disasters or other geopolitical causes. It is worth noting that waste streams are often disproportionately channeled into disadvantaged communities, leading to negative health and environmental impacts on the surrounding populations. Our fermentation technology offers a solution to valorize solid, organic wastes for biohydrogen production as well as a solution for local communities to produce valuable green H₂ from abundant local/municipal waste streams and grow their economy. It also frees a community from fluctuating natural gas and electricity prices and promotes energy security.

Sargassum is a serious threat to global and Atlantic coastal communities, where vast rafts of the seaweed regularly wash ashore, posing threats to local ecosystems and communities. The beaching of large masses of the organism can cause a collapse of the coastal ecosystem as bacteria and other microorganisms consume the decaying seaweed, depleting oxygen in the coastal waters and suffocating fish and other aquatic life. These bacteria also emit the potent and potentially deadly volatile toxin hydrogen sulfide as they decompose the seaweed, posing a serious health threat and preventing public access to beaches until the seaweed can be harvested and disposed of. The necessary removal of this seaweed from beaches thus also incurs waste disposal costs on many small local communities. As such, the efficient valorization and decomposition of this biomass uncovered in this project has the potential to mitigate the costs and dangers surrounding Sargassum, while also lowering the cost of sustainable H₂ and providing clean energy to coastal communities.

### CONCLUSIONS

Economic production of second-generation biofuels or bioproducts hinges on cheap and effective deconstruction, utilization, and conversion of the biomass (i.e., lignocellulose) to a target product. Consolidated bioprocessing combines feedstock hydrolysis, fermentation and product generation in an integrated process, simplifies feedstock processing, and holds immense promise in realizing a sustainable bioeconomy. Through ALE and strain engineering, we have achieved the creation of a new strain (KJCBXint) of the leading CBP microorganism, C. thermocellum, to simultaneously co-utilize cellulose and xylan, and demonstrated about 88% and 80% of DMR corn stover cellulose and xylan utilization, respectively. This is achieved without accumulating soluble, intermediate oligosaccharides which would be inhibitory to soluble glucan and xylan utilization. Utilization of more components of a feedstock using a single species will likely lead to more robust fermentation at high loadings at scale while harnessing more carbon and energy embodied in the feedstock. This study herein demonstrated the feasibility to further unlock the potential of consolidated bioprocessing in ways not achieved thus far.

The embodiments described herein should not necessarily be construed as limited to addressing any of the particular problems or deficiencies discussed herein. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

The provided discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods, and devices useful for the present methods can include a large number of optional composition and processing elements and steps.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A non-naturally occurring Clostridium thermocellum comprising an exogenous gene encoding for β-xylosidase wherein the Clostridium thermocellum metabolizes cellulose and xylan to xylose.

2. The non-naturally occurring Clostridium thermocellum of claim 1 comprising an operon comprising genes xylA, xylB and xylD encoding for β-xylosidases.

3. The non-naturally occurring Clostridium thermocellum of claim 2 wherein xylA has a nucleotide sequence that is greater than 70 percent identical to SEQ ID NO: 1.

4. The non-naturally occurring Clostridium thermocellum of claim 2 wherein xylB has a nucleotide sequence that is greater than 70 percent identical to SEQ ID NO: 2.

5. The non-naturally occurring Clostridium thermocellum of claim 2 wherein xylD has a nucleotide sequence that is greater than 70 percent identical to SEQ ID NO: 3.

6. The non-naturally occurring Clostridium thermocellum of claim 2 wherein xylA and xylB are native to Clostridium thermocellum.

7. The non-naturally occurring Clostridium thermocellum of claim 2 wherein xylD is exogenous.

8. The non-naturally occurring Clostridium thermocellum of claim 7 wherein xylD is derived from Thermoanaerobacterium saccharolyticum.

9. The non-naturally occurring Clostridium thermocellum of claim 2 wherein the β-xylosidases reduce xylan degradation intermediates comprising xylose and xylobiose.

10. A method for making biofuel from biomass using a non-naturally occurring Clostridium thermocellum comprising an exogenous gene encoding for β-xylosidase wherein the Clostridium thermocellum metabolizes cellulose and xylan to xylose, wherein the method comprises the step of contacting the non-naturally occurring Clostridium thermocellum with the biomass.

11. The method of claim 10 wherein the non-naturally occurring Clostridium thermocellum comprises an operon comprising genes xylA, xylB and xylD encoding for β-xylosidases wherein xylA has a nucleotide sequence that is greater than 70 percent identical to SEQ ID NO: 1; and wherein xylB has a nucleotide sequence that is greater than 70 percent identical to SEQ ID NO: 2; and wherein xylD has a nucleotide sequence that is greater than 70 percent identical to SEQ ID NO: 3

12. The method of claim 10 wherein the biomass is DMR corn stover cellulose.

13. The method of claim 12 wherein at least 85% of the DMR corn stover cellulose is converted to a sugar that is fermented to a biofuel.

14. The method of claim 10 wherein at least 78% of the xylan is fermented to a biofuel.

15. The method of claim 10 wherein the biofuel is hydrogen.

16. The method of claim 15 wherein the production of hydrogen is 1.8 mol of hydrogen per mole of saccharide.

17. The method of claim 15 wherein the production of hydrogen is 0.46 L H₂/L.
